(19) United States
(12) Patent Application Publication
(10) Pub. No.: US 2017/0002457 A1
(43) Pub. Date: Jan. 5, 2017

(54) COMPOSITES COMPRISING NANOSTRUCTURED DIAMOND AND METAL BORIDE FILMS AND METHODS FOR PRODUCING SAME

(71) Applicant: UAB RESEARCH FOUNDATION, Birmingham, AL (US)

(72) Inventors: Shane A. Catledge, Bessemer, AL (US); Yogesh K. Vohra, Hoover, AL (US); Jamin Johnston, Birmingham, AL (US)

(21) Appl. No.: 15/100,001

(22) PCT Filed: Nov. 26, 2014
(86) PCT No.: PCT/US2014/067548

§ 371 Date: May 27, 2016

Related U.S. Application Data
(60) Provisional application No. 61/909,725, filed on Nov. 27, 2013.

(51) Int. Cl. C23C 16/02; A61F 2/02; A61L 27/30; C23C 16/27; C23C 16/511; A61L 27/04; A61B 17/16; C23C 16/38

(52) U.S. Cl. CPC C23C 16/0272; A61B 17/1615; A61F 2/02; C23C 16/38; C23C 16/0254; C23C 16/27; C23C 16/511; A61L 27/045; A61L 27/303; A61F 2310/00029; A61F 2310/0058; A61F 2310/0067; A61F 2310/00017; A61F 2310/00023; A61F 2310/00035; A61L 2430/02

(57) ABSTRACT

Composites having a substrate, a diamond film, and a metal boride film disposed between the substrate and the diamond film, together with methods for producing the composites.

### CROSS-REFERENCE TO RELATED APPLICATION

This PCT application claims the benefit of priority to U.S. Provisional Patent Application Ser. No. 61/909,725, filed on Nov. 27, 2013, which is hereby incorporated by reference in its entirety.

### STATEMENT OF GOVERNMENT SUPPORT

This invention was made with government support under Grant No. IIP-1317210 awarded by the National Science Foundation. The government has certain rights in the invention.

### FIELD OF INVENTION

The present invention relates to composites comprising nanostructured diamond and metal boride films and methods for producing the same that can be easily utilized in various fields.

### BACKGROUND

The field of nanocrystalline diamond and tetrahedral amorphous carbon films has been the focus of intense experimental activity in recent years, particularly for use in applications such as filed emission display devices, optical windows, and the like. Chemical vapor deposited (CVD) nanocrystalline films have been synthesized from a variety of plasma feed gases, such as hydrogen-rich plasma, fullerene plasma, or methane plasma.

Nanostructured crystalline diamond films are known to provide efficient corrosion and wear protection, and thus, represent a viable coating alternative in various tribological applications. One of the challenges to successfully implementing nanostructured crystalline diamond films in tribological applications lies in the poor adhesion of many chemical-vapor deposited (CVD) diamond coatings to metallic substrates. Substantial progress has been made to overcome some of the challenges associated with traditional diamond film deposition techniques. In particular, the development of nanostructured diamond deposition techniques has enabled the production of ultra smooth diamond films on surfaces (see, for example, U.S. Patent Publication U.S. Pat. No. 6,183,818 and PCT Publication No. WO/2007041381, which are both incorporated by reference herein for the purpose of disclosing production methods for ultra smooth diamond films. Despite developments in this field, the production of well-adhered diamond coatings on materials having a high solubility for carbon remains a challenge. When attempting to deposit diamond coatings on such materials, graphitic carbon can form on the substrate surface (Lawson et al. “Nanostructured Diamond Coated CoCrMo Alloys for Use in Biomedical Implants,” Key Engineering Materials 2005, 284-286, 1015). In cobalt containing alloys, cobalt can act as a catalyst in its reaction with carbon to preferentially form graphite, leading to poor interfacial adhesion of a subsequently grown diamond coating. Attempts by other researchers to either remove cobalt from the surface using acid-etching techniques or to thermally deposit a discrete interlayer to act as a diffusion barrier to cobalt have been met with limited success.

Thus, there remains a need for methods and compositions that overcome these deficiencies and effectively provide films having small average grain size, improved surface smoothness, satisfactory surface adhesion, and/or desirable stability and hardness on materials that exhibit a high solubility for carbon.

### SUMMARY

Disclosed are composites that comprise: a) a substrate, b) a diamond film having a surface roughness in the range of about 14 nm to about 100 nm, and c) an at least partially continuous metal boride layer disposed between the substrate and the diamond film. In a further aspect, the substrate comprises cobalt in an amount from greater than 0 wt. % to about 75 wt. %.

In a yet further aspect, disclosed herein is a biomedical device comprising the disclosed composite. In a still further aspect, disclosed herein is a cutting device that comprises the disclosed composite.

In other aspects, disclosed herein is a film disposed on a substrate, the film comprising an at least a partially continuous metal boride layer that is conformal to the substrate and having an average surface roughness in the range of about 10 nm to about 75 nm.

In another aspect, disclosed herein is a method comprising forming an at least partially continuous metal boride film on a surface of a substrate. In one aspect, the method comprises: a) introducing the substrate into a reaction chamber; b) introducing a first reaction feed gas mixture; and then c) bringing the reaction chamber to conditions effective to react the first reaction feed gas mixture with the substrate to form the at least partially continuous metal boride layer.

In a further aspect, disclosed is a method comprising forming an at least partially continuous metal boride film on a surface of a substrate, and then forming a diamond film. In one aspect, the method further comprises: a) introducing the substrate into a reaction chamber; b) introducing a first reaction feed gas mixture; c) bringing the reaction chamber to conditions effective to react the first reaction feed gas mixture with the substrate to form the at least partially continuous metal boride layer; and d) exposing the at least partially continuous metal boride layer to a second reaction feed gas mixture at conditions effective to form a diamond film, wherein the diamond film is substantially free of graphitic carbon, substantially free of elemental metal, and exhibits a hardness of at least about 50 GPa.

While aspects of the present invention can be described and claimed in a particular statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect.

Additional advantages of the invention will be set forth in the description and figures which follow or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in, and constitute a part of this specification, illustrate several embodiments and together with the description illustrate the disclosed compositions and methods.

FIG. 1 illustrates high-resolution scanning electron microscopy (SEM) images of (a) a microcrystalline diamond film and (b) a nanostructured diamond film.

FIG. 2 illustrates atomic force microscopy (AFM) images (both topographical and phase images) for a microcrystalline diamond and for a nanostructured diamond coating.

FIG. 3 illustrates an exemplary apparatus suitable for use with the disclosed methods.

FIG. 4 illustrates glancing angle X-Ray diffraction (XRD) patterns of a CoCrMo surface after a CVD boriding process: (a) five degree glancing angle XRD illustrates the formation of body-centered tetragonal Co₂B, orthorhombic CoB, orthorhombic CrB (O), body-centered tetragonal CrB (T), and rhombohedral MoB as the surface layer of borided CoCrMo; (b) enhanced detail from 40 to 50 degrees illustrating masking of (111) FCC cobalt at 44.2 degrees.

FIG. 5 illustrates a cross-sectional SEM image (top) and corresponding energy dispersive spectroscopy (EDS, bottom) of a borided CoCrMo disk, wherein the line in the EDS spectrum corresponds to the line in the SEM image.

FIG. 6 illustrates an optical image of scratch testing using a hemispherical diamond tip: (a) 100× magnification; (b) 500× magnification.

FIG. 7 illustrates the: (a) nano-indentation hardness of a diamond film on an untreated CoCrMo substrate; (b) the average hardness measured at 400 nm for a CoCrMo-boride coated substrate; (c) load vs. displacement for an untreated CoCrMo substrate for up to a 250 mN load; and (d) load vs. displacement for a CoCrMo-boride coated substrate for up to 250 mN.

FIG. 8 illustrates a comparison of XRD peak intensity for Co₂B and FCC Co after boriding at: (a) various temperatures for 1 hour, and (b) for various times at a temperature of 750° C.

FIG. 9 illustrates a comparison of XRD peak intensity for Co₂B and FCC Co after boriding at: (a) various temperatures for 1 hour; (b) for various times at a temperature of 750° C.

FIG. 10 illustrates Raman spectra of a nanostructured diamond deposition on CoCrMo (a) with and (b) without a metal boride layer. The inset illustrates an adhered nanostructured diamond coating on (left) coated CoCrMo and (right) uncoated CoCrMo.

FIG. 11 illustrates X-Ray photoelectron spectroscopy (XPS) scans for: (a) an uncoated CoCrMo substrate, (b) the same substrate after CVD boriding, and c) the same substrate after CVD boriding and deposition of a nanostructured diamond coating.

FIG. 12 illustrates AFM images after (a) nanostructured diamond deposition, and (b) CVD boriding.

FIG. 13 illustrates SEM images obtained during experiments using interfacial oxides: (a) before coalescence of a nanostructured diamond film, and (b) of nodular carbides/oxides in the indicated regions of (a).

FIGS. 14 (a) and (b) illustrate a bulk indentation testing setup using a Rockwell Tester.

FIGS. 15 (a), (b), and (c) illustrate pin-on-disk wear after 2 million cycles for polyethylene (PE)-on-nanostructured diamond film and polyethylene (PE)-on-CoCrMo in bovine serum.

FIG. 16 illustrates a top view of carbide bits after industrial drilling operations with and without a nanostructured diamond layer.

FIG. 17 illustrates an edge view of carbide bits after industrial drilling operations with and without a nanostructured diamond layer.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a component,” “a surface,” or “a noble gas” can include mixtures of two or more such components, surfaces, or noble gases, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Volume percent” or “vol. %” means the percentage of the total volume of a composition or mixture due to a particular component. As used herein, the volume percent of a particular component is used with respect to the total volume of all other gaseous components. For the disclosed compositions and methods, it is understood that each component can be present in the disclosed compositions, along with other optional components, in a concentration necessary for the total concentration of all the gaseous components to equal 100 vol. %.

Likewise, “mass percent” or “mass %” means the percentage of the total mass of a composition or mixture due to a particular component. As used herein, the mass percent of a particular component is used with respect to the total mass of all other gaseous components. For the disclosed compositions and methods, it is understood that each component can be present in the disclosed compositions, along with other optional components, in a concentration necessary for the total concentration of all the gaseous components to equal 100 mass %.

“Optional” or “optionally” means that the subsequently described event or circumstance may or may not occur, and that the description includes instances where said event or circumstance occurs and instances where it does not.

As used herein, the term “substantially” means that the subsequently described event or circumstance completely occurs or that the subsequently described event or circumstance generally, typically, or approximately occurs. For example, when the specification discloses that method steps are performed in a “substantially simultaneous” manner, a person skilled in the relevant art would readily understand that the steps need not be synchronized. Rather, this term conveys to a person skilled in the relevant art that the method steps can be synchronized, can be overlapping in time, or can be separated by a technically insignificant (e.g., commercially insignificant) amount of time. As a further example, when the specification discloses that a composition is “substantially free” of an agent, a person skilled in the relevant art would readily understand that the composition need not be completely free of the agent (i.e., the agent need not be completely absent from the composition). Rather, this term conveys to a person skilled in the relevant art that the agent need only be present in a technically insignificant amount or concentration. In certain aspects, a composition is “substantially free” of an agent when present in less than an amount or concentration less than that necessary to alter the basic and novel properties of the composition. To that end, when an embodiment is described as “substantially free” of a substance, the embodiment can, for example, have no more than 0.1%, 0.2%, 0.5%, 1%, 2%, 3%, 4%, 5%, 6%, or 10% of the substance, relative to the total mass of the embodiment, or in the alternative, relative to the mass of a component (e.g., a layer) thereof.

As used herein, the term “well-adhered” or “substantially adhered” can describe a layer-to-substrate (coating-to-substrate) composite structure wherein substantially no delamination or spalling of the layer from the substrate occurs under a load. In one aspect, no delamination of the layer from the substrate occurs under a load. In a further aspect, substantially no delamination of the layer from the substrate occurs under a load of up to about 60 kg, up to about 100 kg, or up to about 150 kg. In a yet further aspect, substantially no delamination of the layer from the substrate occurs under a load of at least about 60 kg, at least about 100 kg, or at least about 150 kg, for example, from bulk indentation experiments.

In one aspect, the terms “nanostructured diamond film” or “nanocrystalline diamond film” are used interchangeably and can refer to a diamond film having a crystallinity in the range from about 40% to about 75%, for example, about 40, 42, 44, 46, 48, 50, 52, 54, 56, 58, 60, 62, 64, 66, 68, 70, 72, 74, or 75%; from about 50% to about 75%; or from about 60% to about 75%. In another aspect the terms can refer to a diamond film having a grain size in the range from about 5 nm to about 100 nm, for example, about 5, 10, 15, 20, 25, 30, 35, 40, 45, 50, 55, 60, 65, 70, 75, 80, 85, 90, 95, or 100 nm.

As used herein, the terms “diamond-like carbon” or “DLC” can be used interchangeably and are intended to generally refer to highly amorphous, sp²- and sp³-based carbon materials. In one aspect, DLC films can include amorphous carbon (a-C) films and tetrahedral carbon (t-C) films. t-C films typically have a higher content of sp³ carbon than sp² carbon and are typically harder than a-C, with a hardness of up to about 40-60 GPa. Diamond-like carbon films do not contain diamond crystallites and are, therefore, distinct from diamond layers, which are typically fabricated by using plasma-based or hot-filament deposition. DLC films are known to have high residual stress (up to 10 GPa), which can result in poor adhesion on steels, carbides, and other materials, and can also prevent the growth of thick films.

As used herein, the term “orthopedic implant,” is intended to refer to any device that can be placed into the body by any means available in the art to restore function by replacing or reinforcing damaged structures, such as bone, or organs.

As used herein, the term or phrase “effective,” “effective amount,” or “conditions effective to” refers to such amount or condition that is capable of performing the function or property for which an effective amount is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the terms “reference composite” or “reference device” refer to a composite or a device that is substantially identical to the inventive composition, including substantially the same proportions and components but in the absence of an inventive components. In an exemplary aspect, and without limitation, a reference composite or device can have a substantially identical shape and/or size, and utilize the same substrate material as an inventive composite or device but in the absence of an inventive film deposited on the substrate. In another exemplary aspect, a reference composite or device can have a substantially identical shape and/or size, utilize the same substrate material, and have at least one of the substantially identical films deposited on the substrate, but in the absence of combination of other inventive films.

Disclosed are components to be used to prepare the disclosed compositions, as well as the compositions themselves to be used with the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures, which can perform the same function which are related to the disclosed structures, and that these structures will typically achieve the same result.

### B. COMPOSITES

In one aspect, the disclosure provides a composite comprising: a) a substrate; b) a diamond film having a surface roughness in the range of about 14 nm to about 100 nm; and c) an at least partially continuous metal boride layer disposed between the substrate and the diamond film. In another aspect, the disclosed composite exhibits improved wear and corrosion resistance when compared to a reference composite, such as a substrate coated with a diamond film and not having a metal boride layer disposed therebetween.

In one aspect, the disclosed composite can exhibit a hardness that is at least about 5 times greater than the hardness of a reference composite without the diamond film and/or an the at least partially continuous metal boride layer. In a further aspect, the composite structure can have a hardness that is at least about 50 GPa, at least about 60 GPa, at least about 70 GPa, at least about 80 GPa, at least about 90 GPa, or at least about 100 GPa. In another aspect, the composite structure can have a hardness of about 50 GPa to about 100 GPa, for example, about 50, 55, 60, 65, 70, 75, 80, 85, 90, 95, or 100 GPa; or from about 50 GPa to about 80 GPa, for example, about 50, 60, 70, or 80 GPa.

In one aspect, the at least partially continuous metal boride layer can be disposed on the entire surface of the substrate. In a further aspect the at least partially continuous metal boride layer can be disposed on a portion of the surface of a substrate.

In one aspect, the substrate can comprise silicon, metal carbide, or a metal substrate. In one aspect, the substrate is a metal substrate. In one aspect, the substrate can comprise at least one of zirconium, titanium, tungsten, aluminum, molybdenum, vanadium, niobium, cobalt, chromium, silicon, silicon oxide, aluminum oxide, zirconium oxide, or titanium oxide, tungsten oxide, or a mixture thereof, or an alloy thereof. In a further aspect, the substrate or a surface thereof can comprise at least one of Co, Ni, and Fe. In a further aspect, the substrate can comprise an alloy. For example, the substrate can comprise at least one of Ti-6Al-4V, Ti-13Nb-13Zr, CoCr, CoCrMo, a steel, or a mixture thereof.

In another aspect, the substrate can comprise cobalt. In another aspect, the substrate can comprises a cobalt containing alloy. In one aspect, the substrate comprises cobalt in an amount from greater than 0% by weight to about 75% by weight, including exemplary amounts of about 0.05% by weight, about 0.5% by weight, about 1% by weight, about 2% by weight, about 3% by weight, about 4% by weight, about 5% by weight, about 6% by weight, about 7% by weight, about 8% by weight, about 9% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, and about 70% by weight. In another aspect, cobalt can be present in any range derived from any two values set forth above. For example, cobalt can be present in an amount in the range from about 0.05% by weight to about 10% by weight, about 6% by weight to about 25% by weight, or about 10% by weight to about 75% by weight.

In one aspect, the substrate can further comprise one or more of chromium, molybdenum, tungsten, titanium, aluminum, vanadium, nickel, iron, manganese, carbon, or any combination thereof.

In another aspect, the substrate is a metal carbide. In yet another aspect, the metal carbide can comprise tungsten carbide, titanium carbide, or any combination thereof.

In one aspect, the composite disclosed herein comprises a diamond film that is positioned in at least partial overlying registration with at least a portion of the at least partially continuous metal boride layer.

In some aspects, the diamond film is a uniform film. In other aspects, the diamond film has a uniform thickness throughout the film. In yet other aspects, the diamond film has a uniform surface roughness. In one aspect, the diamond film can have a surface roughness in the range of from about 14 nm to about 100 nm, including exemplary roughness values of about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, and about 95 nm. In a further aspect, the diamond film can have a surface roughness in any range derived from any two values set forth above. For example, the surface roughness can be in the range from about 14 nm to about 30 nm, or about 20 nm to about 80 nm, or about 15 nm to about to 100 nm.

In yet another aspect, the diamond film can exhibit a surface roughness of about 14 nm or less, about 12 nm or less, about 10 nm or less, or about 5 nm. In a further aspect, the diamond film can exhibit a surface roughness of about 5 nm or more, about 10 nm or more, about 12 nm or more, or about 14 nm.

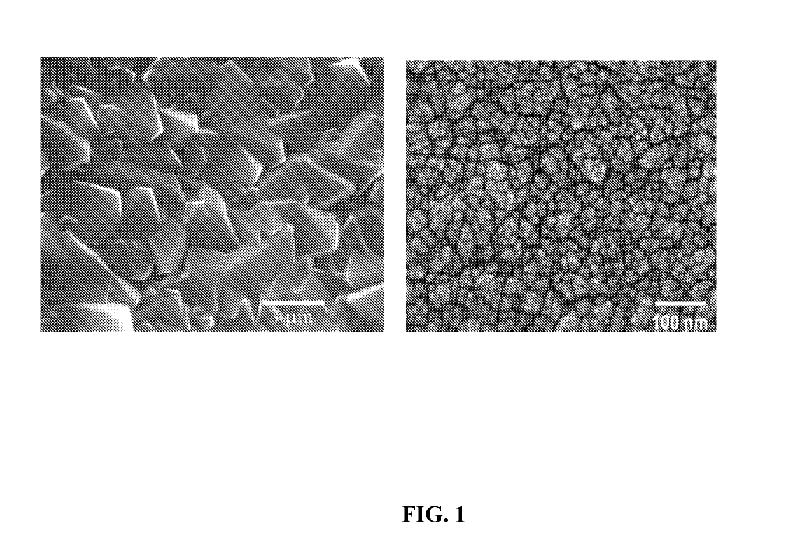

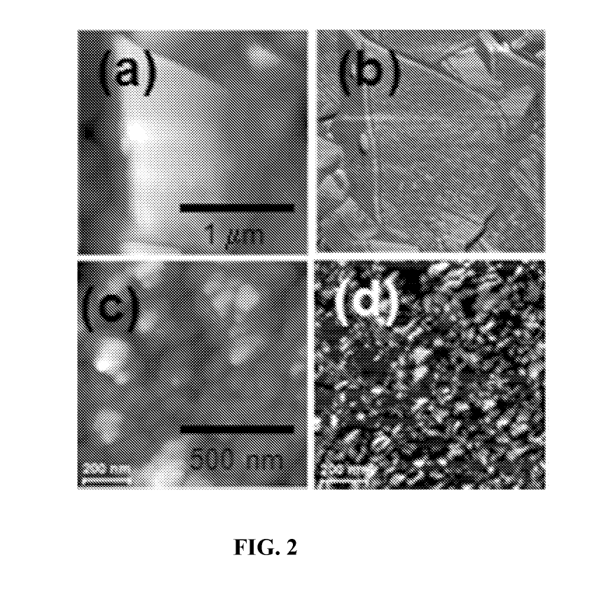

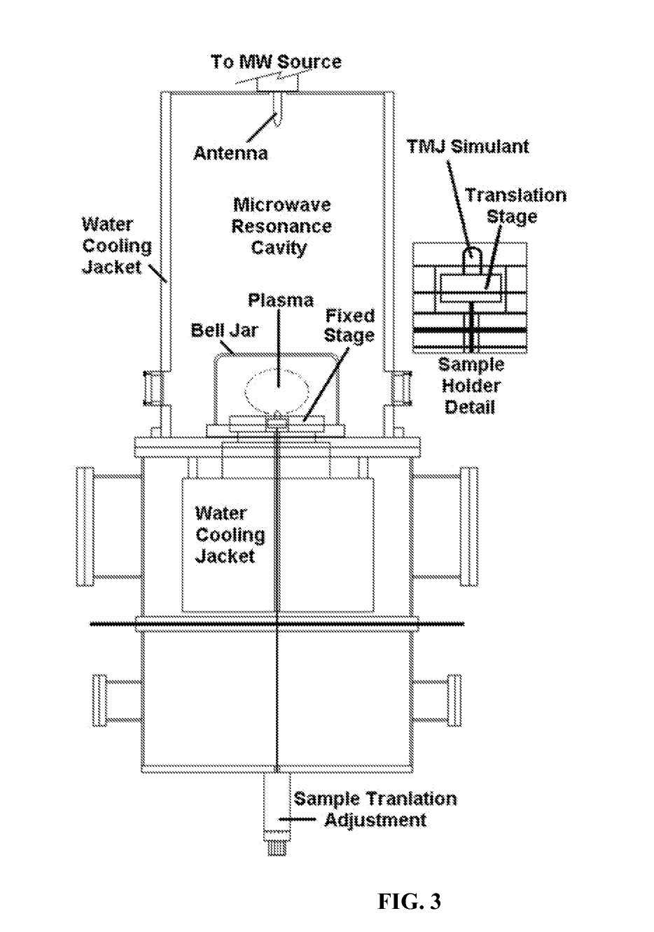

In another aspect, the diamond film can have a surface roughness in the range of about 5 nm to about 100 nm and is an ultra smooth diamond film. In yet another aspect, the diamond film is a nanostructured diamond film. FIG. 1 illustrates high resolution scanning electron micrographs of microcrystalline (left) and nanostructured (right) diamond films. FIG. 2 illustrates topographic (a and c) atomic force microscopy images and corresponding phase images (b and d) for microcrystalline and nanocrystalline diamond films, respectively. The nanostructured diamond films reveal domains of different contrast that are associated with a secondary structure such as variations in carbon bonding, hardness, friction, and the like. Without wishing to be bound by any theory, it is believed that this secondary structure is due to the inherent property differences between diamond and amorphous carbon.

In one aspect, the disclosed composite structures can comprise ultra smooth nanostructured diamond films. The disclosed films can generally exhibit an average grain size of less than about 30 nm, for example, less than 20 nm, less than 15 nm, less than 10 nm, less than 8 nm, or less than 5 nm.

In a further aspect, the nanostructured diamond film can have an average grain size of from about 5 nm to about 100 nm, for example, from about 15 nm to about 100 nm, or from about 15 nm to about 30 nm, and an average surface roughness of from about 15 nm to about 30 nm, for example, or from about 15 nm to about 30 nm. In one aspect, the films can have an average grain size of about 10 nm.

In a further aspect, the nanostructured diamond can have an average grain size of from about 3 nm to about 100 nm, for example, from about 5 nm to about 100 nm, from about 10 nm to about 50 nm, or from about 3 nm to about 30 nm; an average surface roughness of from about 5 nm to about 20 nm, for example, from about 5 nm to about 10 or from about 8 nm to about 10.

In yet another aspect, the nanostructured diamond can have a relative diamond crystallinity of at least about 40%, at least about 50%, or at least about 60%. In another aspect, the nanostructured diamond can have a relative diamond crystallinity of from about 40% to about 75%, for example, about 40, 45, 50, 55, 60, 65, 70, or 75%. In yet another aspect, the nanostructured diamond can have a relative diamond crystallinity up to about 60% or up to about 75%.

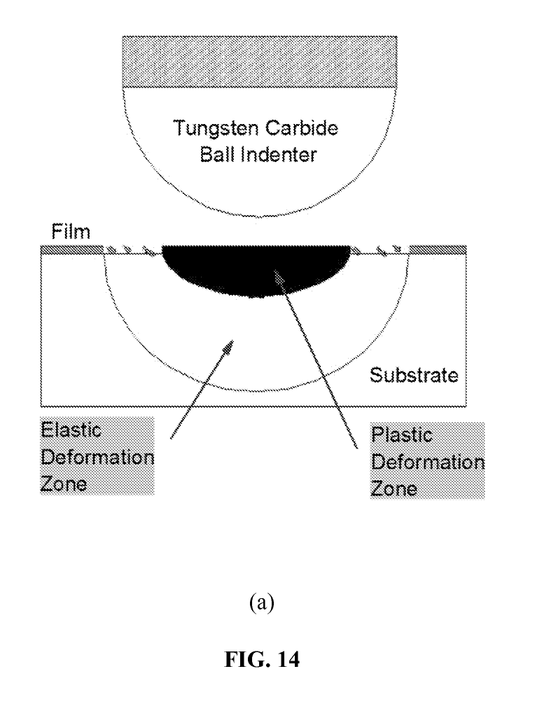

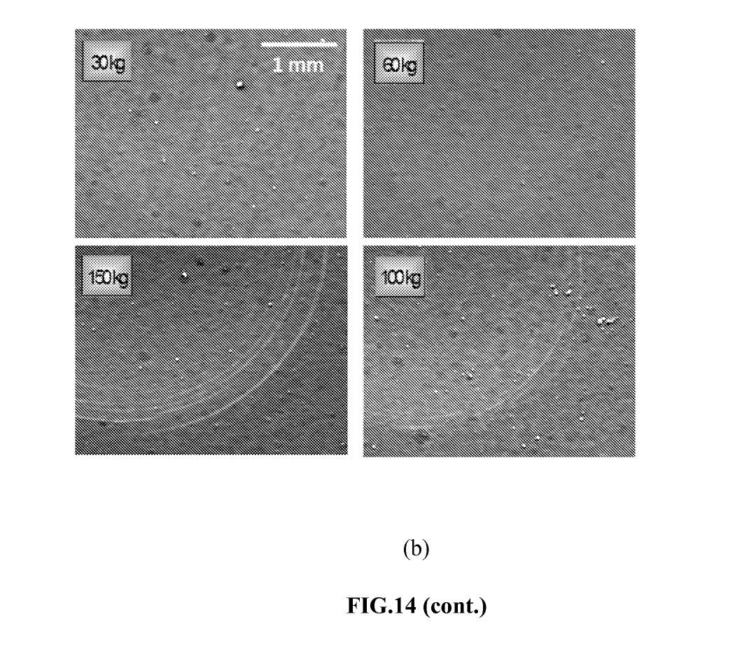

In a further aspect, the nanostructured diamond remains adhered after an indentation load of from about 15 kg to about 150 kg is applied to the composite structure. In one aspect, the adherence of a nanostructured diamond film can be determined using an indentation tester, such as, for example, a Rockwell indentation tester, as illustrated in FIG. 14.

As one of ordinary skill in the art would readily appreciate, transition metal borides exhibit little solubility with carbon and have densely packed structures that can effectively inhibit carbon diffusion. In such an aspect, where the substrate is borided steel, carbon can diffuse away from the boride layer and form borocementite. In one aspect, a nanostructured diamond film adheres to the at least partially continuous metal boride layer. In another aspect, the nanostructured diamond film is at least partially chemically bonded to the at least partially continuous metal boride layer.

In one aspect, the diamond film of the present invention is substantially free of graphitic carbon. In a further aspect, the surface of the substrate is substantially free of graphitic carbon. In a still further aspect, the nanostructured diamond film is free of graphitic carbon. In yet a further aspect, the substrate is free of graphitic carbon. In a further aspect, the entire composite structure is substantially free of graphitic carbon. In another aspect the diamond film is substantially free of an elemental metal.

In another aspect, the diamond film of the present invention exhibits a hardness up to about 80% of the hardness exhibited by a single crystal diamond, including exemplary values of up to about 10%, up to about 20%, up to about 30%, up to about 40%, up to about 50%, up to about 60%, up to about 70%, and up to about 80% of the hardness exhibited by a single crystal diamond.

In one aspect, the at least partially continuous metal boride layer present in the composition that is disposed between the substrate and the diamond film has a thickness of about 15 μm or less, including exemplary values of about 14 μm or less, about 13 μm or less, about 12 μm or less, about 11 μm or less, about 10 μm or less, about 9 μm or less, about 8 μm or less, about 7 μm or less, about 6 μm or less, about 5 μm or less about 4 μm or less, about 3 μm or less, about 2 μm or less, and about 1 μm or less. In another aspect, the at least partially continuous metal boride layer has a thickens of greater than 0 μm, about 1 μm or more, about 2 μm or more, about 3 μm or more, about 4 μm or more, about 5 μm or more, about 6 μm or more, about 7 μm or more, about 8 μm or more, about 9 μm or more, about 10 μm or more, about 11 μm or more, about 12 μm or more, about 13 μm or more, about 14 μm or more, and about 15 μm.

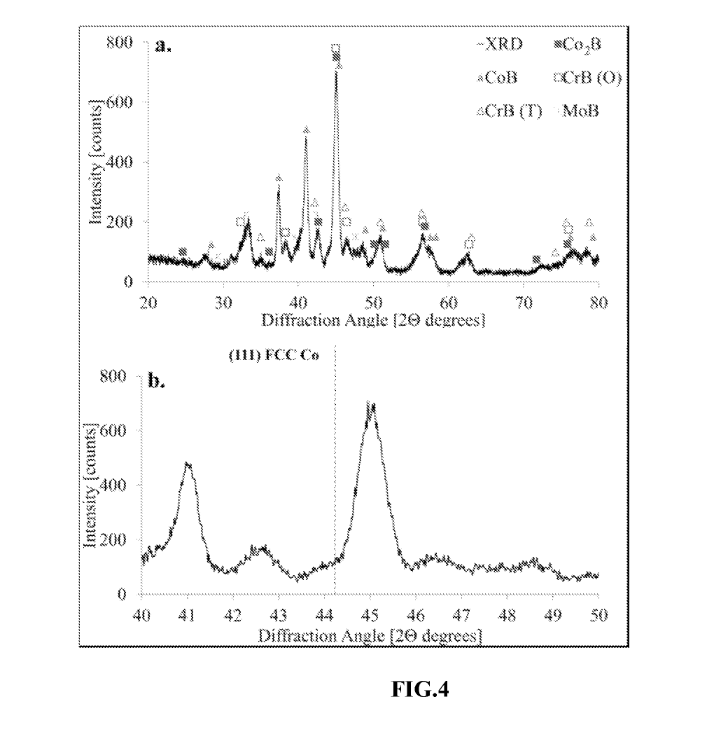

In one aspect, the at least partially continuous metal boride layer comprises cobalt boride. In another aspect, cobalt boride present in the at least partially continuous metal boride layer comprises each of a Co₂B and a CoB phase in predetermined ratio. Without wishing to be bound by any theory, and as supported by FIG. 4, the at least partially continuous metal boride layer can comprise body-centered Co₂B. It can be further seen that when additional metals are present in the substrate, other boride compounds can be formed and can comprise orthorhombic CrB (O), body-centered tetragonal CrB (T), and rhombohedral MoB compounds. In one aspect, the ratio of Co₂B to CoB can be in the range from about 0:100 to about 50:50. In still other aspects, other cobalt tungsten boride compounds, such as, for example, CoWB, CoW₂B₂, or a combination thereof can be formed.

In one aspect, the at least partially continuous metal boride layer is conformal to the surface of the substrate or a portion thereof. In another aspect, the at least partially continuous metal boride layer is conformal to any shape of the substrate. In one aspect, the substrate can have any shape useful for a specific application. In another aspect, the substrate shape can be easily determined by one of ordinary skill in the art.

In one aspect, the at least partially continuous metal boride layer is substantially free of elemental boron. In another aspect, any cobalt present in the at least partially continuous metal boride layer is chemically bound to boron. In another aspect, the at least partially continuous metal boride layer is substantially free of unbound cobalt. In some aspect, the at least partially continuous metal boride layer has an average surface roughness in the range of about 10 nm to about 75 nm, including exemplary values of about 15 nm, about 20 nm, about 25 nm, about 30 nm, about 35 nm, about 40 nm, about 45 nm, about 50 nm, about 55 nm, about 60 nm, about 65 nm, and about 70 nm. In a further aspect, the at least partially continuous metal boride layer can have a surface roughness in any range derived from any two values set forth above. For example, the surface roughness can be in the range from about 15 nm to about 30 nm, or about 20 nm to about 50 nm, or about 20 nm to about 70 nm.

In one aspect, the at least partially continuous metal boride layer can exhibit an average hardness of from about 5 GPa to about 50 GPa, including exemplary values of about 10 GPa, about 12 GPa, about 15 GPa, about 18 GPa, about 20 GPa, about 22 GPa, about 25 GPa, about 27 GPa, about 30 GPa, about 32 GPa, about 35 GPa, about 38 GPa, about 42 GPa, about 45 GPa, and about 50 GPa. In another aspect, a boride metal layer can have a hardness of up to about 25 GPa, up to about 35 GPa, or up to about 50 GPa. In yet another aspect, a metal boride layer can have a hardness of at least about 20 GPa, at least about 25 GPa, or at least about 30 GPa. In a further aspect, the at least partially continuous metal boride layer can have exhibit an average hardness in any range derived from any two values set forth above. For example, the average hardness can be in the range from about 5 GPa to about 32 GPa, or about 10 GPa to about 25 GPa, or about 15 GPa to about 38 GPa.

### C. METHODS

Chemical vapor deposited (CVD) diamond films grown using gas mixtures such as hydrogen, nitrogen, and methane have been previously used to form smooth nanocrystalline diamond films. [S. A. Catledge and Y. K. Vohra, J. Appl. Phys. 84, 6469 (1998); S. A. Catledge, J. Borham, Y. K. Vohra, W. R. Lacefield, and J. E. Lemons, J. Appl. Phys. 91, 5347 (2002); A. Afzal, C. A. Rego, W. Ahmed, and R. I. Cherry, Diam. Rel. Mater. 7, 1033 (1998); R. B. Corvin, J. G. Harrison, S. A. Catledge, and Y. K. Vohra, Appl. Phys. Lett. 84, 2550 (2002).] A film grown without nitrogen addition typically shows large, well defined crystalline facets indicative of high-phase-purity diamond. [S. A. Catledge and Y. K. Vohra, J. Appl. Phys. 83, 198 (1998).] In contrast, films grown with added nitrogen typically exhibit a nanocrystalline appearance with weak agglomeration into rounded nodules of submicron size. It has also been observed that the transformation from microcrystalline to nanocrystalline diamond structure can occur by adding Ar in H₂/CH₄ feed gases with a total transformation observed at Ar/H₂ volume ratio of 9. [D. Zhou, D. M. Gruen, L. C. Qin, T. G. McCauley, and A. R. Krauss, J. Appl. Phys. 84, 1981 (1998); D. M. Gruen, Annu. Rev. Mater. Sci. 29, 211 (1999).] The effect of helium addition to H₂/CH₄/N₂ feedgas mixtures on the growth of high quality ultra-smooth nanostructured diamond films on Ti-6Al-4V has also been reported. [V. V. Konovalov, A. Melo, S. A Catledge, S. Chowdhury, Y. K. Vohra, J. Nanosci. and Nanotechnol., 6, 258 (2006).] Each of the references listed above are incorporated by reference herein for the purpose of disclosing methods for forming various diamond films.

Many inherent challenges in coating nanostructured diamond onto a metal substrate have been overcome; however, known chemical vapor deposition methods are not sufficient to produce adherent and quality diamond films on materials that exhibit a high solubility for carbon. In particular, well adhered diamond coatings on transition metals such as iron, nickel, or cobalt, cannot be achieved using known methods. While not wishing to be bound by theory, it is believed that this is due to the formation of interfacial graphitic carbon during the deposition process, which precludes strong adhesion between the substrate and the film. The formation of interfacial graphitic carbon is a result of catalytic behavior of metals such as nickel and cobalt. These transitions metals have high carbon diffusivity and do not form stable carbides, making further nucleation of the diamond phase difficult. Once a graphitic layer is formed, the adhesion of a subsequently formed diamond layer can be weak.

The inventive methods disclosed herein are directed to the formation of an efficient barrier layer capable of inhibiting the catalytic behavior of metals such as cobalt. In certain aspects, disclosed herein are methods of forming an at least partially continuous metal boride film on a surface of a substrate. Generally, in one aspect, the disclosed methods can comprise a) introducing the substrate into a reaction chamber; b) introducing a first reaction feed gas mixture in an effective amount to form an at least partially continuous metal boride layer; and then c) bringing the reaction chamber to conditions efficient to react the first reaction feed gas mixture with the substrate to form the at least partially continuous metal boride layer. It should be noted that while certain conditions, for example, temperature, time, and concentration, are recited herein, one of skill in the art, in possession of this disclosure, would be able to determine appropriate reaction conditions.

In one aspect, the reaction chamber comprises a plasma reactor. In another aspect, the plasma reactor is a chemical vapor deposition plasma reactor. In certain aspects, the plasma reactor is a microwave plasma reactor, for example, as illustrated in FIG. 3. It should be understood that plasma reactor configurations are not limited to those disclosed herein and can comprise any plasma reactor or plasma reactor configuration capable of providing chemical vapor deposition of the desired film. As used herein, the term “plasma” refers to any plasma wherein energy is imparted to a gas mixture by any of the usual forms of forming a plasma. A DC arc, an RF discharge, a plasma jet, a capacitive plasma, an inductive plasma, high density plasma, hot wire, a microwave, a laser beam, an electron beam, or a combination thereof can be used as an energy source to create the plasma disclosed herein. While microwave plasma chemical vapor deposition (MPCVD) has been used to describe the plasma source and deposition method, the method is not intended to be limiting and the disclosed compositions, methods, and films can be used in connection with any method for establishing a plasma known to those of skill in the art.

In one example, a microwave plasma enhanced CVD system (ASTeX PDS-17) can be employed for the any disclosed film depositions.

In some aspects, the first reaction feed gas mixture comprises a mixture of diborane (B₂H₆) and hydrogen (H₂) gases. In some aspects, hydrogen gas used in the first reaction feed gas mixture is a high purity gas, exhibiting a purity of greater than about 95%, greater than about 96%, greater than about 97%, greater than about 98%, greater than about 99%, greater than about 99.1%, greater than about 99.2%, greater than about 99.3%, greater than about 99.4%, greater than about 99.5%, greater than about 99.6%, greater than about 99.7%, greater than about 99.8%, greater than about 99.9%, greater than about 99.95%, greater than about 99.99%, or greater than about 99.995%. In other aspects, diborane gas can be provided as a 10% dilution in hydrogen gas.

In various aspects, diborane gas can be provided as a pure gas and be mixed with hydrogen. In another aspect, diborane can be provided as a diluted gas, for example, as 10% diborane in hydrogen, and then optionally be further diluted with the same or a different gas. In one aspect, a diluted diborane gas (e.g., 10% in hydrogen) can be provided at a flow rate of from about 1.0 standard cubic centimeters (sccm) to about 5.0 standard (sccm). In another aspect, such a diluted diborane gas can be provided at a flow rate of about 3.0 sccm. In a further aspect, hydrogen gas can be provided at a flow rate of from about 100 sccm to about 1,000 sccm, or from about 500 sccm to about 1,000 sccm. In a specific aspect, a typical flow can comprise 5 sccm diluted diborane and 1,000 sccm hydrogen. In another specific aspect, hydrogen gas can be provided at a flow rate of about 500 sccm. In certain aspects, diborane and hydrogen gases can be provided in a ratio of B₂H₆/H₂ in the range from about 1:100 to about 1:1,000, including exemplary values of about 1:100, about 1:200, about 1:300, about 1:400, about 1:500, about 1:600, about 1:700, about 1:800, about 1:900, or about 1:1,000. Additionally, or in the alternative, the first reaction feed gas mixture can be provided at a flow rate or at a ratio so as to form conditions effective to deposit a metal boride film.

In certain aspects, prior to step of introducing the first reaction feed gas mixture to the reaction chamber, the substrate placed in the reaction chamber can be exposed to a substantially pure hydrogen gas. In other aspects, the substrate exposed to the substantially pure hydrogen gas is heated. In yet another aspect, the substrate is exposed to the substantially pure hydrogen gas with ignited plasma. In a yet further aspect, the substrate is exposed to the substantially pure hydrogen gas, is heated, and the plasma is ignited. In certain aspects, the substrate is exposed to substantially pure hydrogen gas and heated to reach a steady state temperature.

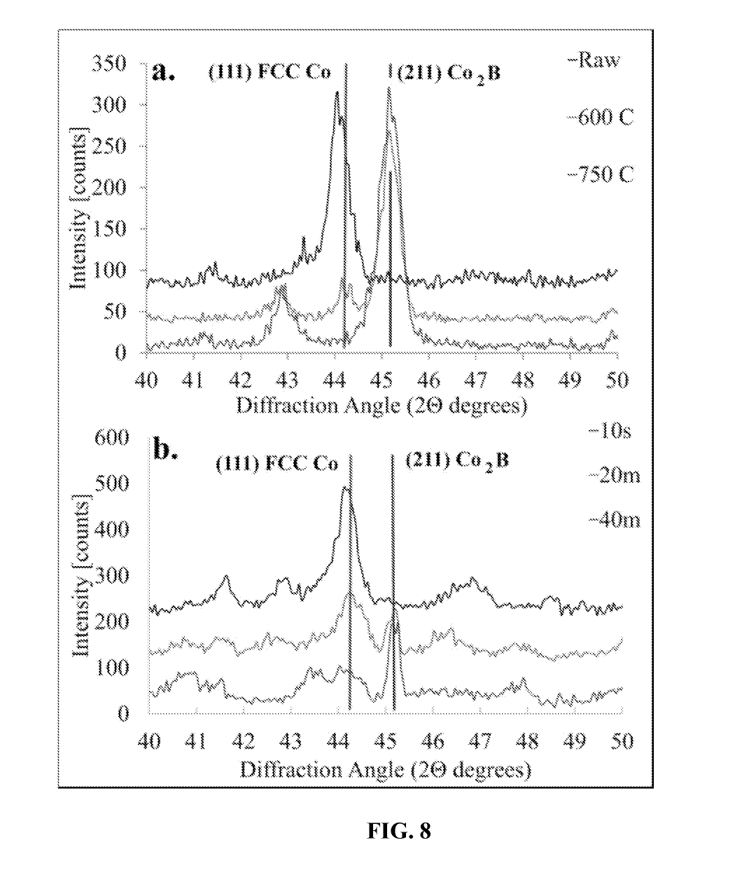

In some aspects, the conditions effective to react the first reaction feed gas with the substrate to form the at least partially continuous metal boride layer comprise igniting plasma. In certain aspects, the plasma can be contained at a pressure in the range from about 20 Torr to about 150 Torr. In some other aspects, plasma power can be in the range from about 0.600 kW to about 3.0 kW. In yet other aspects, the conditions effective to react the first reaction feed gas with the substrate to form the at least partially continuous metal boride layer comprise a temperature in the range of about 500° C. to about 800° C., including exemplary values of about 520° C., about 550° C., about 580° C., about 600° C., about 620° C., about 650° C., about 680° C., about 700° C., about 720° C., about 750° C., and about 780° C. In other aspects, the temperature can be in any range derived from any two values set forth above. For example, the temperature can be in the range of about 550° C. to about 650° C. or about 600° C. to about 750° C. The effect of the temperature on the at least partially continuous metal boride film deposition at the constant time is demonstrated in FIG. 8.

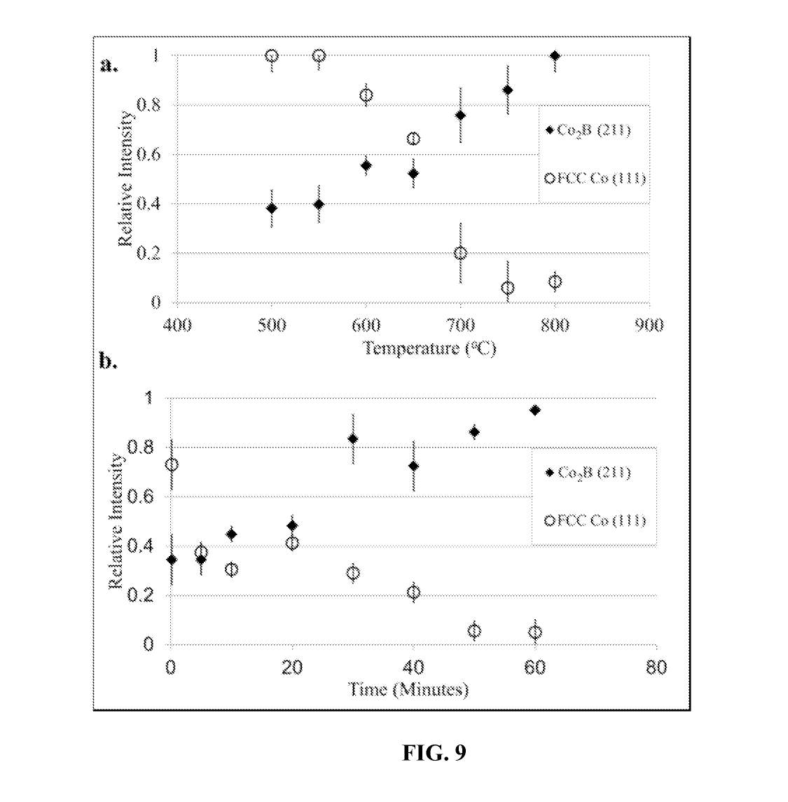

In certain aspects, the conditions effective to react the first reaction feed gas with the substrate to form the at least partially continuous metal boride layer can further comprise exposure of the substrate to the first feed gas mixture plasma for a time period of about 10 seconds to about 2 hours, including exemplary values of about 30 sec, about 1 min, about 5 min, about 10 min, about 20 min, about 30 min, about 40 min, about 50 min, about 1 hour, about 1 hour 10 min, about 1 hour 20 min, about 1 hour 30 min, about 1 hour 40 min, and about 1 hour 50 min. In other aspects, the exposure time can be any time in any range derived from any two values set forth above. For example, the exposure time can be in the range of about 30 sec to about 1 hour or about 1 hour to about 2 hours. The effect of time on the at least partially continuous metal boride film deposition at the constant temperature is illustrated in FIG. 9.

In some aspect, the substrate used to form the metal boride films can be any substrate disclosed above. In one aspect, the at least partially continuous metal boride layer can be disposed on a surface of the substrate. In one aspect, a portion of the surface of the substrate can be covered with a “mask” prior to deposition of the at least partially continuous metal boride layer, wherein after deposition the “mask” can be removed, providing a patterned film on the portion(s) of the surface of the substrate. Optionally, before deposition, the surface of the substrate can be prepared to receive the disclosed films by polishing or abrading to ensure a satisfactory starting surface smoothness. For example, the surface of the substrate can be polished by one of many methods known to those of skill in the art, for example, mechanical polishing with fine powder, such as diamond, silica, or alumina; chemical-mechanical polishing; chemical etching; solid state diffusion; or abrading the surface using varying grit sandpapers.

In a yet further aspect, optionally, before deposition, the surface of the substrate can be modified by creating surface defects. For example, the surface can be modified by scratching or sand blasting.

In some aspects, the polished surface of the substrate can be further cleaned by any means known in the art prior to introducing the substrate into the reaction chamber. For example and without limitation, cleaning can comprise rinsing the surface of the substrate in organic, inorganic, aqueous or nonaqueous solvents. In exemplary aspects, the solvents can include but are not limited to acetone, methanol, or deionized water. The cleaning can be accompanied by mechanical mixing, sonication, heating, or cooling, and the required step can be easily determined by one of ordinary skill in the art. In an even further aspect, optionally, before deposition, the surface can be prepared, pre-treated, and/or modified using one or more of the above-described techniques.

In certain aspects, the at least partially continuous metal boride layer deposited by the inventive methods has a thickness of about 15 μm or less. In some aspects, the inventive methods disclosed herein allow formation of the at least partially continuous metal boride layer that exhibits cobalt diffusion barrier properties and is at least 10 times thinner than a boride film produced by conventional pack boriding methods and having a thickness of about 100 to about 200 μm. As one of ordinary skill in the art would readily appreciate, the use of chemical vapor deposition, as compared to conventional pack boriding, allows the formation of a film that is easily conformal to the original shape and edge sharpness of a substrate.

Without wishing to be bound by theory, it is hypothesized that the use of the inventive first reaction feed gas mixture allows the formation of an at least partially continuous metal boride layer near the surface of the substrate that is expected to be resistant to diffusion of elemental cobalt towards the surface, to be mechanically robust, and to partially compensate for interfacial and film stresses due to thermal expansion differences between the substrate and a subsequently grown nanostructured diamond film. It is further hypothesized that the at least partially continuous metal boride film can have an irregular interfacial morphology that can aid adhesion of the boride layer to the substrate by creating mechanical “interlocking” effects. Additionally, high levels of physical intermixing and chemical reactions between the components of the film and the substrate can also affect film adhesion.

FIG. 4 illustrates glancing angle XRD patterns of a CoCrMo substrate after a CVD metal boride layer deposition. As one of ordinary skill in the art would readily appreciate, the XRD patterns reveal that the at least partially continuous metal boride layer comprises body-centered tetragonal Co₂B and orthorhombic CoB phases, orthorhombic CrB (O), body-centered tetragonal CrB (T), and rhombohedral MoB phases. It should be noted that other metal borides, such as, for example, CoWB, CoW₂B₂, or a combination thereof can be formed, for example, when cemented tungsten carbide is borided. FIG. 4b focuses on a narrower 40-50 degrees range to enhance scan detail where FCC Co (111) would be observed. The FCC cobalt (111) peak at 44.2 degrees, that is dominant in the unborided alloy, is masked at the surface after boriding, demonstrating that boriding provides a surface that is substantially free of unbound elemental metal. Without wishing to be bound by theory, it is believed that the lack of elemental cobalt indicates the potential of the boride layer as a diffusion barrier for subsequent nanostructured diamond film growth. In certain aspects, the body-centered tetragonal Co₂B and orthorhombic CoB phases are present in a predetermined ratio. As one of ordinary skill in the art would readily appreciate, the inventive CVD based deposition method can allow better control of a desired metal-boride stoichiometry. Without wishing to be bound by theory, it is believed that films with higher content of the Co₂B phase relative to the CoB phase, are desirable. The CoB phase is expected to be more brittle and susceptible to cracking. In certain aspects, the predetermined ratio of Co₂B to CoB can be in the range of from about 0:100 to about 50:50. It should be understood that this ratio is exemplary and not intended to be limiting. As one of ordinary skill in the art would readily appreciate, the ratio of Co₂B to CoB can be dependent on the substrate utilized for the deposition.

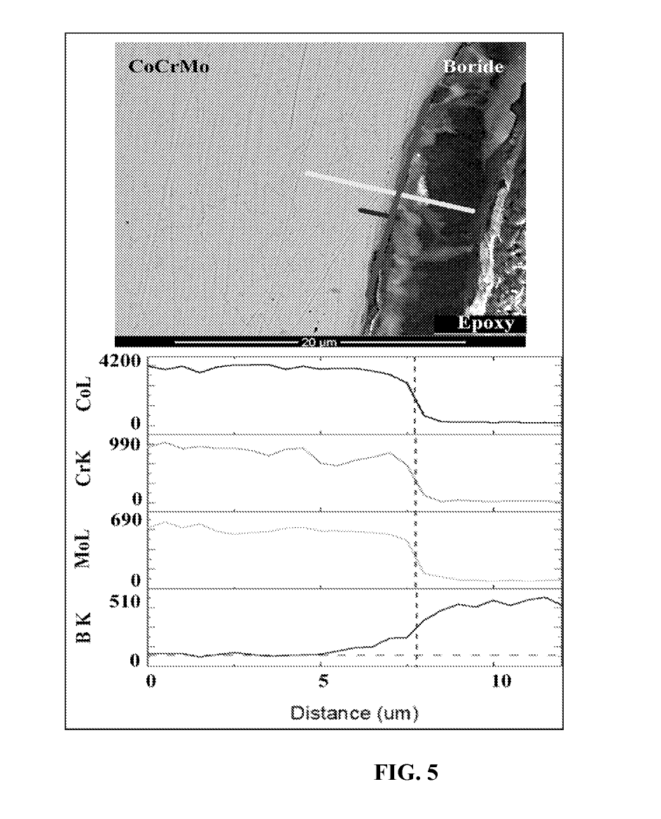

SEM images and EDS spectra of a borided CoCrMo substrate are illustrated in FIG. 5. EDS analysis provides a qualitative determination of the spatial variation in primary elements (Co, Cr, and Mo) from the surface to the bulk substrate. It can be seen that the boride layer effectively suppresses metal migration to the surface. Without wishing to be bound by theory, it is hypothesized that diffusion of cobalt towards the surface and through a dense boride layer would occur predominantly by a vacancy-assisted mechanism. The activation energy for vacancy exchange in the covalently-bonded boride layer is expected to be sufficiently high compared to the alloy to limit such diffusion. Formation of these covalent boride compounds is also expected to minimize access of elemental cobalt from the bulk to the surface where it could otherwise interact with carbon to form graphite during subsequent nanostructured diamond film deposition.

In certain aspects, the at least partially continuous metal boride layer formed by the disclosed inventive methods is substantially free of elemental boron. In other aspects, any cobalt present in the at least partially continuous metal boride layer is chemically bound to boron. In still further aspects, the at least partially continuous metal boride layer is substantially free of unbound cobalt. In yet other aspects, the at least partially continuous metal boride layer is substantially free of contaminations. In some aspects, the at least partially continuous metal boride layer can have any surface roughness disclosed above. In exemplary aspect, the at least partially continuous metal boride layer can have a surface roughness in the range of from about 10 nm to about 75 nm.

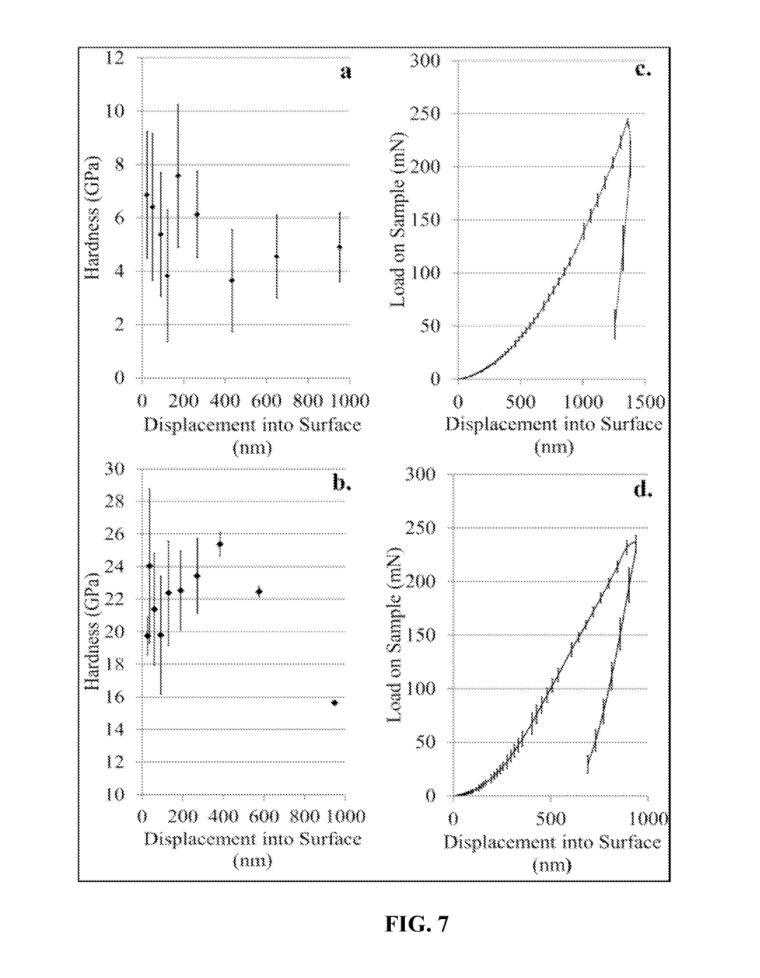

As one of ordinary skill in the art would readily appreciate, high levels of internal stress can cause coating delamination, and the internal stress can be greatest at the coating/substrate interface. The nanoidentation load-displacement behavior of a composite can provide a measure of the elastic and plastic deformation contributions from the indentation. Tougher coatings will result in a larger plastic depth contribution (W_{plastic}) from the indentation load vs. displacement curve. The nanoidentation data is shown on FIG. 7. In some aspects, the at least partially continuous metal boride layer formed by the disclosed inventive methods, for example, on a CoCrMo substrate, can exhibit an average hardness of from about 5 GPa to about 25 GPa. In other aspects, a borided tungsten carbide can exhibit an average hardness of up to about 30 GPa, 40 GPa, or 50 GPa.

In certain aspects, the methods disclosed herein can further comprise exposing the at least partially continuous metal boride layer to a second reaction feed gas mixture at conditions effective to form a diamond film. In some aspects, prior to the exposure to the second reaction feed gas mixture, the substrate with the at least partially continuous metal boride layer can be removed from the reaction chamber. In other aspects, the removed boride layer can be exposed to a cleaning step. As one of ordinary skill in the art would readily appreciate the cleaning step can comprise any cleaning methods known in the art, including but are not limited to rinsing with organic, inorganic, aqueous, nonaqueous solvents, or any combinations thereof. In exemplary aspects, the cleaning can include rinsing with acetone, rinsing with methanol and/or rinsing with deionized water. In certain aspects, the cleaned metal boride layer can be dried, wherein drying can be any drying method known in the art.

In certain aspects, the substrate with at least partially continuous metal boride layer can be returned to the reaction chamber after cleaning. In other aspects, the metal boride layer deposited on the substrate can be returned to the reaction chamber after drying. In certain aspects, the cleaned and/or dried metal boride layer can be returned to the same or a different reaction chamber. In an exemplary aspect, a borided substrate can be ultrasonically seeded with, for example, a diamond nanoparticle slurry prior to cleaning and re-introducing into a reaction chamber. In such an aspect, scratches and/or defects can be created that enhance nucleation of diamond formed from the CVD process.

In certain aspects, the second reaction feedgas mixture can comprise a mixture of methane and hydrogen. In some other aspects, the second reaction feed gas mixture can further comprise nitrogen. In some aspects, a ratio of methane to hydrogen is in the range of about 1:6 to about 1:20, including exemplary values of about 1:7, about 1:8, about 1:19, about 1:10, about 1:11, about 1:12, about 1:13, about 1:14, about 1:15, about 1:16, about 1:17, about 1:18, and about 1:19. In other aspects, methane to hydrogen can be present in any range derived from any two values set forth above. For example, the ratio of methane to hydrogen can be about 1:7 to about 1:15, or about 1:10 to about 1:20. In yet other aspects, methane can be present in the mixture in amount of about 5 vol. % to about 15 vol. % in a balance of hydrogen, including exemplary values of about 6 vol. %, about 7 vol. %, about 8 vol. %, about 9 vol. %, about 10 vol. %, about 11 vol. %, about 12 vol. %, about 13 vol. %, and about 14 vol. %. In other aspects, diborane can also be present during CVD diamond growth. In one aspect, diborane can added during the initial portion of CVD diamond growth. In another aspect, diborane can be present during the initial 30 minutes of CVD diamond growth. While not wishing to be bound by theory, diborane present during CVD diamond growth can react with any remaining cobalt to prevent upward diffusion of any remaining elemental cobalt.

In some aspects, nitrogen can be present in a concentration of from about 2% to about 20% of methane by volume, including exemplary amounts of about 3%, about 4%, about 5%, about 6%, about 7%, about 8%, about 9%, about 10%, about 11%, about 12%, about 13%, about 14%, about 15%, about 16%, about 17%, about 18%, about 19% by volume. In yet other aspects, nitrogen can be present in any range set forth above. For example, nitrogen can be present in the range of about 5% to about 10% by volume, or about 8% to about 18% by volume.

In one aspect, the compositions can further comprise a noble gas component in a concentration of up to about 90 vol. %. Various noble gasses can be used in the disclosed gas compositions to prepare the disclosed films. In one aspect, the noble gas component can comprise helium, neon, argon, krypton, xenon, radon, or a mixture thereof. In a further aspect, the noble gas component can be helium.

In one aspect, the noble gas component, or mixture of two or more noble gases, is present in the disclosed compositions in a concentration of from about 40 vol. % to about 95 vol. %. For example, the noble gas component can be present at from about 40 vol. % to about 90 vol. %, from about 50 vol. % to about 80 vol. %, from about 60 vol. % to about 70 vol. %, from about 50 vol. % to about 60 vol. %, from about 60 vol. % to about 70 vol. %, from about 70 vol. % to about 80 vol. %, from about 80 vol. % to about 90 vol. %, from about 60 vol. % to about 80 vol. %, or from about 70 vol. % to about 80 vol. %. In a further aspect, the noble gas component is present at from about 25 vol. % to about 93.9 vol. %.

In a further aspect, the noble gas component and nitrogen can be present in the disclosed compositions in a combined concentration of less than about 80 vol. % of the composition. In a yet further aspect, the noble gas component and the nitrogen component can be present in a combined concentration of less than about 75 vol. % of the composition. For example, the noble gas component and nitrogen can be present at from about 40 vol. % to about 80 vol. %, from about 40 vol. % to about 75 vol. %, from about 45 vol. % to about 75 vol. %, from about 50 vol. % to about 70 vol. %, from about 55 vol. % to about 65 vol. %, from about 60 vol. % to about 70 vol. %, from about 65 vol. % to about 75 vol. %, from about 70 vol. % to about 75 vol. %, from about 75 vol. % to about 80 vol. %, from about 65 vol. % to about 75 vol. %, or from about 70 vol. % to about 80 vol. %.

In a yet further aspect, mixtures of two or more noble gasses can be used in the disclosed compositions to perform the disclosed methods and/or to prepare the disclosed films. For example, the noble gas component can be present as a mixture of from about 1 vol. % to about 99 vol. % helium and from about 99 vol. % to about 1 vol. % argon, for example, as a mixture of from about 10 vol. % to about 90 vol. % helium and from about 90 vol. % to about 10 vol. % argon, from about 20 vol. % to about 80 vol. % helium and from about 80 vol. % to about 20 vol. % argon, from about 30 vol. % to about 70 vol. % helium and from about 70 vol. % to about 30 vol. % argon, from about 40 vol. % to about 60 vol. % helium and from about 60 vol. % to about 40 vol. % argon, or as about 50 vol. % helium and about 50 vol. % argon. It should be understood that other noble gasses (for example, neon, krypton, xenon, and/or radon) can be added to or substituted for helium and/or argon in the disclosed mixtures, compositions, and methods. Volume percent (i.e., vol. %) can be expressed in terms of a total functional gaseous composition volume, or in the alternative, in terms of a total gaseous composition volume.

In certain aspects, the conditions effective to form the diamond film further comprise igniting a plasma by microwave discharge to provide a chemical vapor deposition of the diamond film. In some aspects, the conditions effective to form the diamond film comprise running the plasma process under a pressure of from about 30 Torr to about 150 Torr, including exemplary values of about 35 Torr, about 40 Torr, about 45 Torr, about 50 Torr, about 55 Torr, about 60 Torr, about 65 Torr, about 70 Torr, and about 75 Torr. In yet other aspects, the conditions effective to form the diamond film comprise running the plasma process under a pressure in any range derived from any two values set forth above. For example, the conditions effective to form the diamond film comprise running the plasma process under a pressure from about 30 Torr to about 120 Torr, or about 45 Torr to about 140 Torr.

In yet other aspects, the conditions effective to form the diamond film comprise running the plasma process under a substrate temperature of from about 700° C. to about 850° C., including exemplary values of about 705° C., about 710° C., about 715° C., about 720° C., about 725° C., about 730° C., about 735° C., about 740° C., about 745° C., about 750° C., about 755° C., about 760° C., about 765° C., about 770° C., about 775° C., about 780° C., about 785° C., about 790° C., about 795° C., about 800° C., about 805° C., about 810° C., about 815° C., about 820° C., about 825° C., about 830° C., about 835° C., about 840° C., and about 845° C. In yet other aspects, the conditions effective to form a diamond film comprise running the plasma process at a temperature in any range derived from any two values set forth above. For example, the conditions effective to form the diamond film comprise running the plasma process at a temperature from about 700° C. to about 750° C., or about 730° C. to about 800° C.

In certain aspects, the diamond films are nanostructured diamond films. In some aspects, the diamond films formed by the inventive methods can have any thickness disclosed above. In other aspects, the diamond films formed by the inventive methods can have an average surface roughness in the range of about 14 nm to about 100 nm. In yet other aspects, the diamond films have an average grain size of from about 3 nm to about 100 nm. In certain aspects, the diamond films are adhered to at least a portion of the at least partially continuous metal boride layer. In some aspects, the diamond film is at least partially chemically bonded to the at least partially continuous metal boride. In other aspects, the diamond film is substantially free of a graphitic carbon. In yet other aspects, the diamond film is substantially free of an elemental metal. In still other aspects, the diamond film is substantially free of elemental boron.

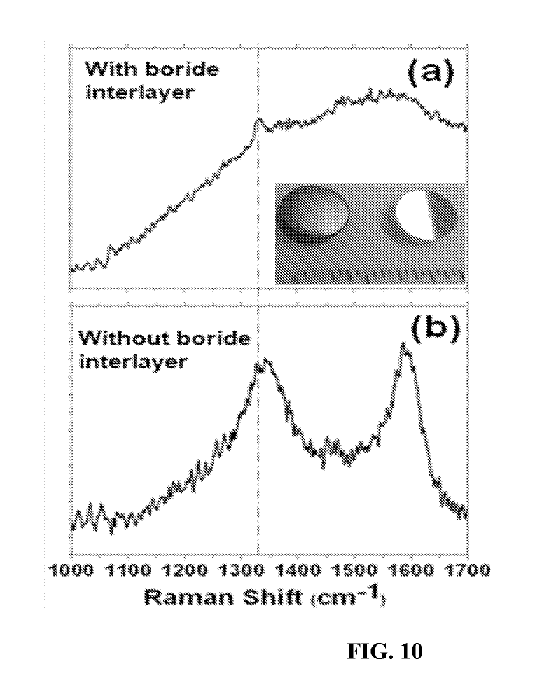

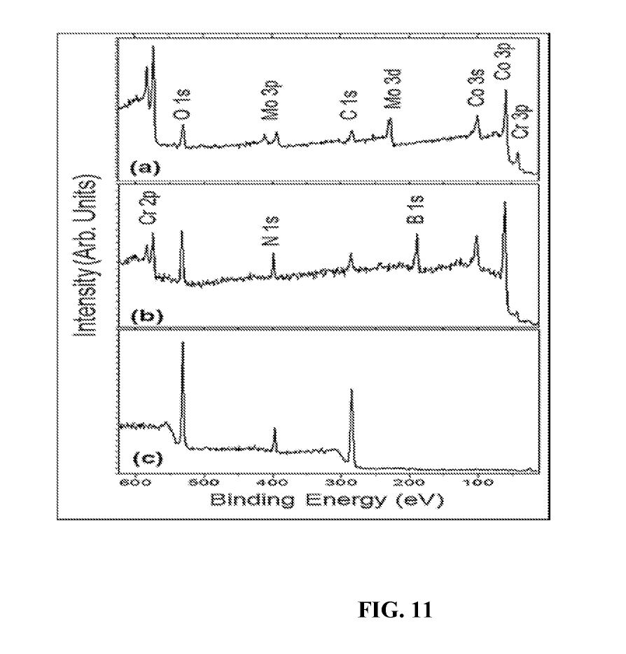

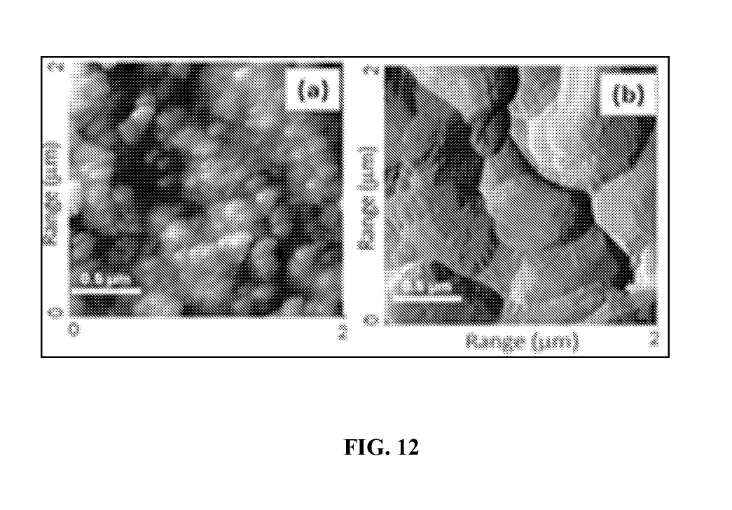

FIG. 11 illustrates XPS spectra performed on (a) untreated, (b) CVD-borided, and (c) nanostructured diamond coated (after boriding) CoCrMo surfaces, as shown in survey scans (0-600 eV). As one of ordinary skill in the art would readily appreciate, migration of molybdenum can be suppressed after boriding, and does not appear on the surface. The high-resolution B1s spectrum (not shown) indicates that, in one aspect, most boron is present as borides (peak c.a. 188.5 eV) with very small contributions from boron nitrides (191.0 eV) and oxides (192.5 eV). In other aspects, chromium can be present both in elemental form on the surface and as chromium nitrides/oxides. Depositing nanostructured diamond over the boride layer (using a H₂/CH₄/N₂ feedgas mixture) can result in a well-adhered coating with surface morphology and Raman spectra characteristic of typical nanostructured diamond structure (FIGS. 10 and 12). The Raman feature at 1332 cm⁻¹ (shown with dashed line) can be attributed to ordered sp³-bonded carbon and the broad band c.a. 1550 cm⁻¹ is associated with disordered carbon. Without use of a boride interfacial layer, microcrystalline graphite can be formed (FIG. 10) with characteristic ‘D’ and ‘G’ bands c.a. 1350 cm⁻¹ and 1580 cm⁻¹, respectively. XPS of the nanostructured diamond coated surface (FIG. 11) reveals only carbon, nitrogen and oxygen.

In certain aspects, the diamond film formed by the inventive methods has a hardness of up to about 80% of the hardness of a single crystal diamond. In yet other aspects, the diamond film has a hardness that is at least about 50 GPa.

Relative diamond crystallinity is a measure of the ratio of sp³ nanocrystalline diamond content to sp²/sp³ amorphous carbon content in the nanostructured diamond films. Relative diamond crystallinity is related to the hardness of the film as well as to the surface adhesion of the film. Generally, the greater the relative diamond crystallinity, the greater the hardness. Also, in conventional films, the greater the relative diamond crystallinity, the less satisfactory the surface adhesion. Relative diamond crystallinity can be measured by XRD analysis. The disclosed nanostructured films formed by the inventive methods can generally have from about 40% to about 75% relative diamond crystallinity. The partially non-crystalline amorphous composition of the nanostructured films is primarily very hard, tetrahedral-coordinated amorphous carbon with small sp²-bonded clusters, or other hard sp² or sp³ carbon amorphous matrix. Without wishing to be bound by theory, it is believed that this amorphous carbon content in the nanostructured diamond film can improve fracture toughness of the films by limiting crack nucleation and by reducing the stress near existing cracks. Therefore, the excellent interfacial adhesion observed for these inventive films (in comparison to crystalline, nanocrystalline, or ultra-nanocrystalline diamond films) can be attributed to a reduction of residual film stress along with an increase in interfacial toughness.

In one aspect, the diamond films deposited by the disclosed methods can have a relative diamond crystallinity of at least about 40%, for example, a relative diamond crystallinity of at least about 40%, of at least about 50%, of at least about 60%, or of at least about 70%. In a yet further aspect, the films can have a relative diamond crystallinity of up to about 70%, for example, of up to about 60%, for example, of up to about 50%, for example, of up to about 40%, for example, or of up to about 30%. In a further aspect, the films can have a relative diamond crystallinity of from about 30% to about 70%, for example, from about 40% to about 60%, from about 30% to about 50%, from about 50% to about 70%, or about 50%.

In one aspect, the inventive method comprises forming an at least partially continuous metal boride film on a surface of a substrate, and then forming a diamond film on the metal boride film. In another aspect, the method further comprises a) introducing the substrate into a reaction chamber, b) introducing a first reaction feed gas mixture in an effective amount to form an at least partially continuous metal boride layer, c) bringing the reaction chamber to conditions effective to react the first reaction feed gas mixture with the substrate to form the at least partially continuous metal boride layer, and d) exposing the at least partially continuous metal boride layer to a second reaction feed gas mixture at conditions effective to form an diamond film, wherein the diamond film is substantially free of graphitic carbon, substantially free of metal, and exhibits hardness of at least 50 GPa.

It should be noted that the substrate materials, gases, and other components described in this application are commercially available and that one of skill in the art, in possession of this disclosure, could readily procure such materials and perform the disclosed methods.

### D. APPLICATIONS

In various aspects, the disclosed films and composites can be used to produce abrasion resistant materials and devices, such as, for example, cutting devices; low wear rate coatings on biomedical devices and implants; high thermal conductivity, high temperature substrates for high power electronic circuits; wide diamond-coated wafers for electronic, optoelectronic, and optical devices; high temperature, ultra-high frequency, high power, high radiation, high-stability transistors; wide optical range windows, wear resistant optical windows; substrates for surface acoustic wave devices; low corrosion, high electrode potential window substrates (electrodes) for biological and/or chemical sensors; and substrates for microelectromechanical or nanoelectromechanical systems (MEMS/NEMS) devices.

In one aspect, a biomedical device, such as an orthopedic implant can comprise the composite, wherein the substrate can comprise a cobalt containing alloy, coated with a metal boride layer, and then with a nanostructured diamond film. In one aspect, such an implant can comprise an artificial knee or a portion thereof. In other aspects, the disclosed films formed by inventive methods can be used to produce coated medical instruments or implants. In various aspects, the orthopedic implants comprising the disclosed composites can provide high hardness, low friction, and wear-resistant behavior, and can be used under severe physiological conditions. The orthopedic medical implants can include, but are not limited to, a femoral head implant, a hip socket implant, a knee implant, a plate, or portions thereof. It should be noted that cobalt containing alloys can provide improved strength for such implants, but the bioavailability of cobalt metal can be of concern. In such aspects, the metal boride layer and nanostructured diamond films of the present invention can inhibit or eliminate the bioavailability of cobalt.

In further aspects, the disclosed composites can be used to produce coated magnetic storage media. In yet further aspects, the disclosed composites and films can be used to produce coated recording heads in magnetic storage media.

In a yet further aspect, a cutting device, such as, for example, a drill bit, can comprise the disclosed composite.

### E. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Sample Preparation

CoCrMo samples (ASTM F1537 low carbon alloy) having a primary composition (by weight) of 28% of Cr, 6% of Mo, and balance of Co were used for boriding and diamond depositions. In some aspects, up to 1% of Si, Mn, and Ni can be present in the alloy. In other aspects, up to 0.75% of Fe and 0.14% of carbon can be present in the alloy. The as-received cylindrical CoCrMo rods were cut into disks by way of electrical discharge machining. The disks measured 7 mm in diameter and 1 mm in height. Disks were polished at 350 rpm for 10 minutes with 200, 400, 600, and 1200 grit wet silicon carbide paper, and then polished with diamond solutions at 9, 6, 3, and 1 microns. Polished samples were then cleaned via sonication with acetone and then methanol for 20 minutes each, followed by a deionized water rinse.

2. Film Boriding Procedure

2.1 Boriding

A 2.45 GHz microwave-plasma CVD (Wavemat, Ann Arbor, Mich., USA) chamber was used for boriding. A schematic representation of the reaction chamber is illustrated in FIG. 3. Boriding was done prior to diamond deposition using a diborane (B₂H₆) and hydrogen (H₂) feedgas mixture. This feedgas mixture was 500 sccm of high purity 99.9% H₂ and 3.0 sccm of B₂H₆ (provided as a 10% dilution in H₂). Pressure and power were incrementally increased until the desired trial specific parameters were met. The deposition pressure was in the range from about 35 Torr to about 55 Torr. The deposition power was in the range from about 0.600 kW to about 1.0 kW. Substrate temperature was measured using a two-color optical pyrometer centered at 1.6 μm. The CoCrMo substrate was first allowed to reach a steady state temperature in H₂, at which point the combined H₂ and B₂H₆ feedgas mixture was introduced. Several CoCrMo samples were produced for a range of target temperatures (500-800° C. at 50-degree intervals) using a one hour duration and for a range of boriding times (10 seconds to 120 minutes) using a 750° C. substrate temperature.

2.2 Nanostructured Diamond Growth

Borided samples were cleaned via sonication for 10 minutes in acetone, 10 minutes in methanol, and then rinsed in deionized water. In order to increase the nucleation density for subsequent nanostructured diamond growth, the borided substrates were sonicated for 20 minutes in a methanol/nanodiamond slurry (International Technology Center, Research Triangle Park, N.C., USA) with average diamond particle size of 4 nm (0.2% w/v). Samples were then rinsed in deionized water and air-dried.

To produce the nanostructured diamond films, microwave plasma chemical vapor deposition was used. The gas flow remained constant for each run at 500 standard cubic centimeters per minute (sccm) of hydrogen gas (H₂, 99.9% purity), 5 sccm nitrogen (N2, 99.9% purity), and 30 sccm methane gas (CH₄, 99.9% purity). The microwave power was controlled in the range from about 1 kW to about 0.6 kW. Chamber pressured was kept at about 40 Torr.

2.3 Boriding and Nanostructured Diamond Growth on Tungsten Carbide Bits

Three samples of tungsten carbide bits were placed in a plasma reactor and a metal boride film was CVD deposited, followed by CVD deposition of a nanostructured diamond film. The tungsten carbide bits were treated for 1 hr at 700° C. in the microwave plasma enhanced chemical vapor deposition with a diborane/hydrogen gas mixture, with the hydrogen flow rate kept at 500 sccm, and diborane gas (diluted in hydrogen) kept at 3 sccm.

After metal boride film deposition, the samples were removed and cleaned in acetone, followed by a rinse in methanol, and then a rinse in deionized water. The cleaned and dried tungsten carbide bits were then exposed to microwave plasma enhanced chemical vapor deposition utilizing methane, hydrogen and nitrogen feedgases at 700° C. The hydrogen flow rate was kept at 500 sccm, the nitrogen flow rate was kept at 50 sccm, and the methane flow rate was kept at 100 sccm. After 1 hour of deposition, the samples were removed and cleaned first in acetone, rinsed in methanol, and subsequently rinsed in deionized water. The dried samples were placed into the reaction chamber for the second diamond film deposition. The second deposition was performed for 1 hour at the same disclosed conditions.

3. Characterization

X-Ray Diffraction (XRD) patterns were measured using a thin-film diffractometer (X'pert MPD, Philips, Eindhoven, The Netherlands) with Cu anode (λ=0.154154 nm), generator voltage of 45 kV, tube current of 40 KA, and glancing angle of 3° and 5°. Scans were compared against the JCPDS (Joint Committee on Powder Diffraction Standards) database and diffraction simulations using CrystalDiffract software. The topography of the coatings was imaged using Atomic Force Microscopy (AFM) and Scanning Electron Microscopy (SEM) with chemical analysis by Energy Dispersive x-ray Spectroscopy (EDS). AFM images were obtained using contact mode and the roughness values were recorded. SEM/EDS measurements were done using an FEI Quanta® 650 FEG. SEM images were captured in secondary electron mode and EDS line scans were performed on the cross-section of the boride layer to observe change in chemical composition across the interface. To prepare samples for cross-sectional analysis, borided discs were mounted in an epoxy resin, cut with a diamond saw, and sanded/polished to a mirror finish using diamond paste. Micro-Raman spectra were collected from the diamond coatings using an argon-ion laser (λ=514.5 nm) at a laser power of 100 mW. Nanoindentation was performed using a Nanoindenter XP system (MTS Systems, Oak Ridge, Tenn.) to evaluate hardness up to a maximum force of 250 mN. Nanoindentation was performed on the bare alloy and on the surface layer after boriding to evaluate changes in hardness and elastic vs. plastic depth. Progressive load scratch tests were performed to observe the extent of cracking or delamination of the coatings. Scratch tests were performed with a commercial diamond stylometer (Romulus IV, Quad Group Inc., Spokane, Wash., USA) with a 125 μm radius spherical diamond tip. The maximum force used was 10 N with a load rate of 5 N/s over a distance of 5 mm. The scratches were examined using an optical microscope.

In order to probe the surface chemistry of the boride and nanostructured diamond layers, X-ray Photoelectron Spectroscopy (XPS) was performed. This PHI Versaprobe imaging XPS was operated using a monochromatic, focused Al Kα X-ray source (E=1486.6 eV) at 25 W with a 100 μm spot size. Charge neutralization was provided by a cold cathode electron flood source and low-energy Ar ions. All measurements were taken at room temperature and at an argon working pressure of 2×10⁻⁶ Pa; the system base pressure was 5×10⁻⁸ Pa. Survey scans were taken at 187.4 eV pass energy, with a 0.8 eV step; high-resolution scans were taken at 23.5 eV pass energy, with a 0.2 eV step. In order to remove surface contamination, the samples underwent 2 min of Ar-ion sputter etching at 500 V accelerating voltage; cratering effects were limited by rastering the ion beam across a 2×2 mm² area.

3.1 CVD Boriding on CoCrMo

FIG. 4a illustrates the XRD spectra of borided CoCrMo for a substrate temperature of 750° C. and boriding time of 60 minutes. It was found that microwave plasma CVD boriding can lead to the formation of a number of various boride compounds. The crystallinity data demonstrates that in addition to body-centered tetragonal Co₂B and orthorhombic CoB phases that were previously observed in powder-pack boriding, the inventive methods can also lead to formation of orthorhombic CrB (O), body-centered tetragonal CrB (T), and rhombohedral MoB. FIG. 4b focuses on a narrower 40-50 degrees range to enhance scan detail where FCC Co (111) would be observed (dashed red line). As one of ordinary skill in the art would readily appreciate, the FCC cobalt (111) peak at 44.2 degrees that is dominant in the unborided alloy, is masked at the surface after boriding, demonstrating that boriding provides a surface that is substantially free of unbound metal.

FIG. 5 demonstrates the extent of boron diffusion using cross-sectional EDS and SEM. Without wishing to be bound by theory, it is speculated that a dense surface layer (dark region) shown on the micrographs includes various metal borides that can be present in the film (based on the XRD spectra shown in FIG. 4). The EDS measurements demonstrate graded boron diffusion into the bulk CoCrMo substrate. Without wishing to be bound by theory, diffusion of boron into the substrate is expected to form chemical compounds with cobalt, resulting in Co₂B and CoB structures. It further hypothesized that formation of these covalent boride compounds (and others) is expected to minimize access of elemental cobalt from the bulk to the surface where it could otherwise interact with carbon to form graphite during subsequent nanostructured diamond deposition.

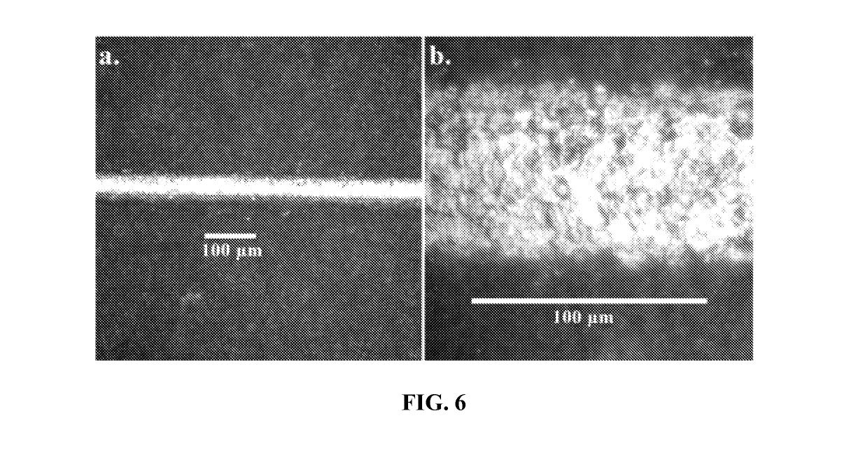

Average boride surface roughness was measured over a 25 μm×25 μm area by AFM to be about 50 nm with a maximum peak-to-valley height of 717 nm. Scratch testing of the borided surface (FIGS. 6a and 6b) showed no signs of extensive cracking or interface delamination up to the maximum normal force of 10 N. The diamond stylus appears to plow through the boride rather than show abrupt elastic-to-brittle failure.

3.2 CoCrMo-Boride Nanoindentation Hardness

Nanoindentation has been used to evaluate the hardness and elastic vs. plastic depth for both un-borided (but polished) CoCrMo and borided CoCrMo and results are shown on FIG. 7. The data with error bars represent average and standard deviation for 10 indents. Tests on the un-borided alloy to 1500 nm depth showed an average hardness of 5.8 GPa. Without wishing to be bound by theory, it is speculated that the peak hardness value can be a result of higher CoB concentration near the surface, with hardness decreasing at depth with increasing concentration of Co₂B near the bulk alloy/boride. The surface of the borided alloy showed significantly increased hardness, peaking to 25.2 GPa near 400 nm depth, and dropping off with increasing depths into the bulk alloy. FIGS. 7c and 7d show average load-displacement data comparing un-borided and borided alloy. For the un-borided alloy, the elastic contribution is approximately 17% while that for the borided alloy is approximately 73%. The change in inflection near 600 nm depth for the loading curve of the borided alloy can be indicative of change in stoichiometry from CoB-rich (harder) to Co₂B-rich (softer), as expected in going from depths closer to the surface to those closer to the boride/alloy interface.

3.3 Borided CoCrMo: Effect of Temperature and Time

FIG. 8a shows XRD of two samples borided at different temperatures (for same amount of time) along with that of a “raw” unborided (control) CoCrMo sample. It was found that an increased temperature yields improved blocking of elemental cobalt (reduced peak intensity at 44.2 degrees) and increased formation of cobalt borides (increased intensity at 45.1 degrees). FIG. 8b shows that for an increased deposition time at 750° C., a continual decrease of FCC cobalt intensity is observed in identical XRD surface scans, indicating a thicker cobalt-masking boride layer. Peak intensity for a given set of XRD scan conditions is directly proportional to crystalline phase concentration within the irradiated volume. A comparison of XRD peak intensity across a range of temperatures (FIG. 9a) and times (FIG. 9b) shows that as temperature or time increase the relative peak intensity caused by surface concentration of elemental cobalt was reduced. Additionally, the presence of Co2B increased. Error has been calculated using the standard of deviation (n=3) at each temperature and time. The presence of FCC cobalt was effectively masked at 60 minutes and 750° C. Likewise, the presence of Co2B was maximized at 60 minutes and 800° C. Temperatures over 750° C. often resulted in inhomogeneous surface deposition and surface layer delamination. Data taken at 120 minutes revealed no significant change from 60 minutes. These results indicate that boriding masked the presence of elemental cobalt and provided a sufficient layer of Co₂B that may be useful for subsequent nanostructured diamond film growth.

FIG. 10 illustrates Raman spectra of the nanostructured film deposited at previously disclosed conditions. The Raman feature at 1332 cm⁻¹ (shown with dashed line) can be attributed to ordered sp³-bonded carbon and the broad band c.a. 1550 cm⁻¹ is associated with disordered carbon (FIG. 10a). Without use of a boride interfacial layer, microcrystalline graphite is formed with characteristic D and G bands c.a. 1350 cm⁻¹ and 1580 cm⁻¹, respectively (FIG. 10b).

3.4 CVD NSD Growth on Borided CoCrMo

FIG. 11 shows the XPS survey spectrum of the film that was done on (a) untreated, (b) CVD borided, and (c) nanostructured diamond film (NSD)-coated (after boriding) CoCrMo surfaces. It was found that migration of molybdenum is suppressed after boriding, and does not appear on the surface. The high-resolution B1s spectrum for CVD-borided CoCrMo (not shown) indicates that most boron is present as borides (peak c.a. 188.5 eV) with very small contributions from boron nitrides (191.0 eV) and oxides (192.5 eV). XPS of the nanostructured diamond film coated surface (FIG. 10c) reveals only carbon, nitrogen and oxygen. No boron or cobalt is found on the nanostructured diamond surface.

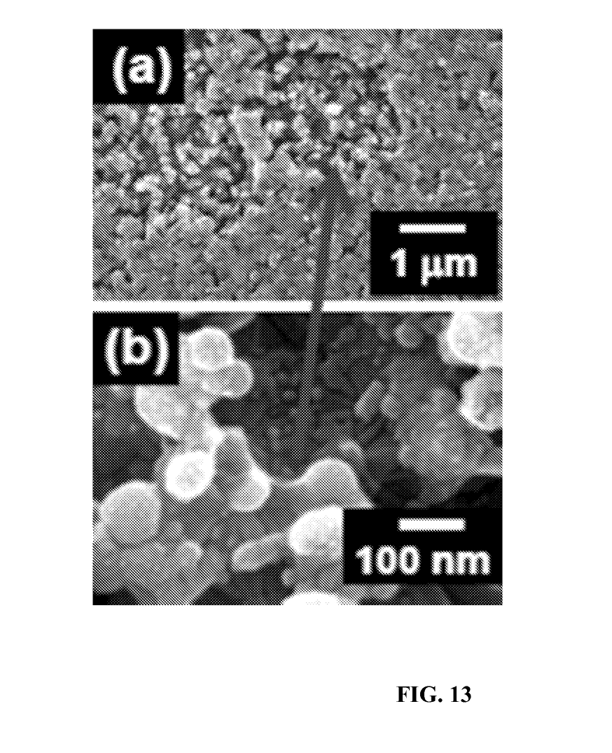

FIG. 12 shows AFM images of the nanostructured diamond deposited on the boride layer. The early stages of nanostructured diamond nucleation/growth have been investigated by the stopping CVD deposition before coalescence of a continuous NSD film and results are shown on FIG. 13.

4. Wear-Resistant Surfaces for Orthopedic Implants

Flat disk samples of the biomedical alloy CoCrMo were obtained and treated accordingly to the above disclosed depositions methods. It is known in the art that common Total Knee Replacement (TKR) implants used in the orthopedic industry contain Ultra-High Molecular Weight Polyethylene (UHMWPE) as a tibial bearing surface. CoCrMo alloy has been the standard material for femoral components in TKR for more than 40 years; however, clinical evidence shows that the surfaces of retrieved CoCrMo femoral components exhibit scratches and roughening that can significantly increase the wear of polyethylene.

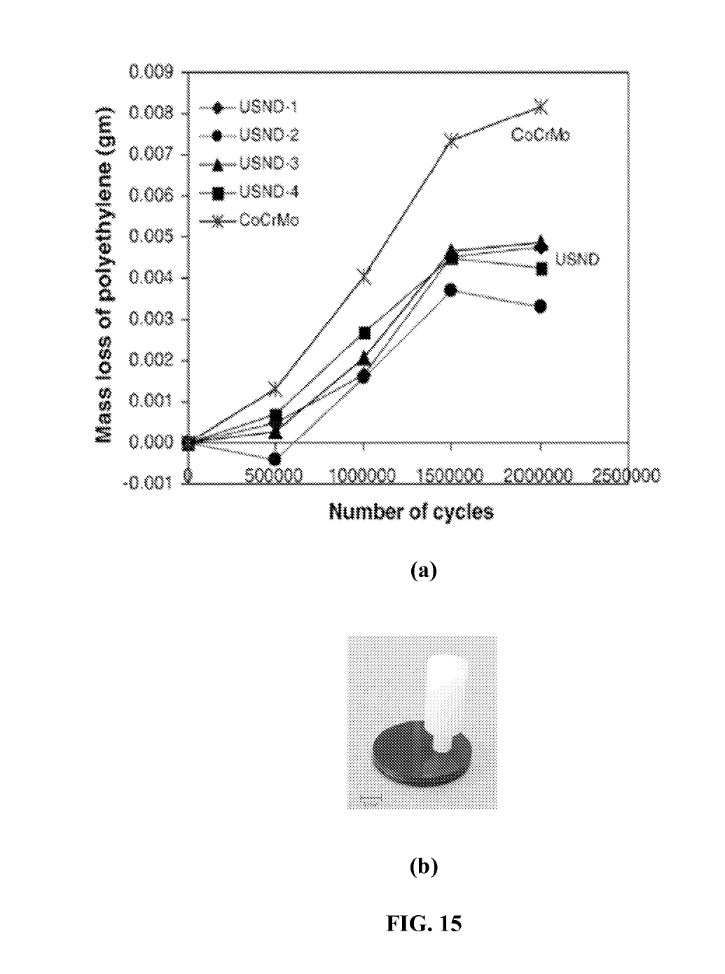

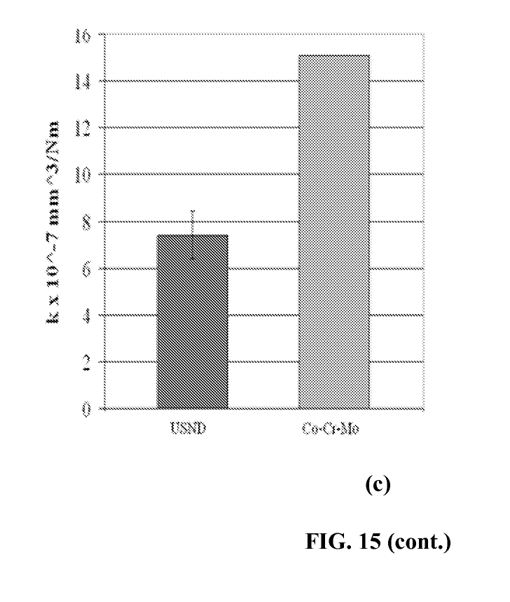

FIG. 15 shows pin-on-disk (polyethylene on nanostructured diamond film) wear data with comparison to polyethylene-on-CoCrMo to two million cycles in calf serum at 37° C. The average wear factor calculated for polyethylene-on-CoCrMo (k=5.7±0.8×10 mm³/Nm) falls within the range determined from retrieval studies of total hip replacements (k=9.0×10⁸ to 7.2×10⁻⁶ mm³/Nm) and is nearly a factor of two higher than that calculated for polyethylene-on-nanostructured diamond film.

5. Tungsten Carbide Bits Drilling Performance

Commercially available cemented tungsten carbide roof bits containing from 6 wt. % to 10 wt. % cobalt as a binder phase (Bama Mine & Milling, Inc., Hueytown, Ala., USA) were treated accordingly to the above disclosed depositions methods.

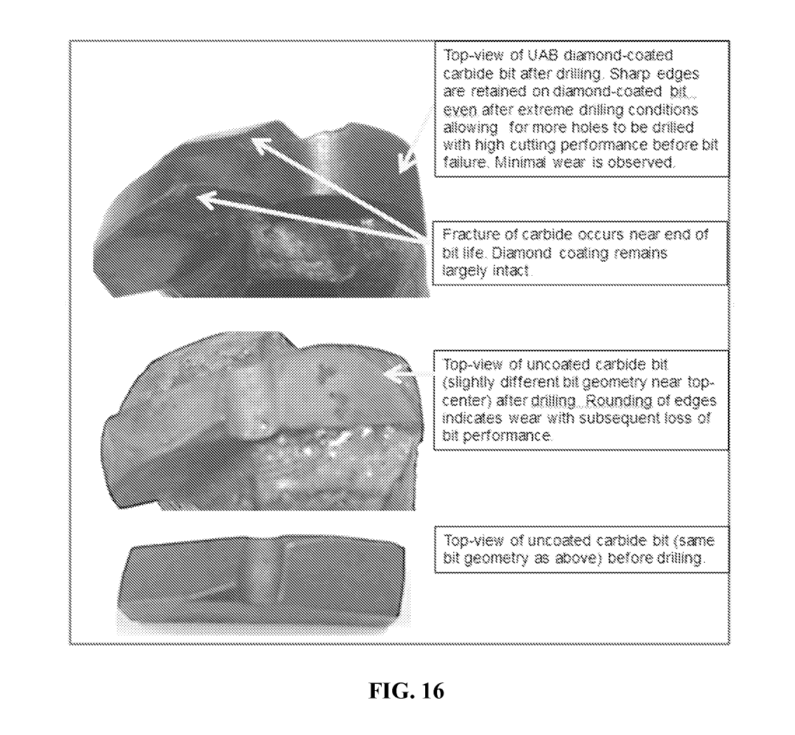

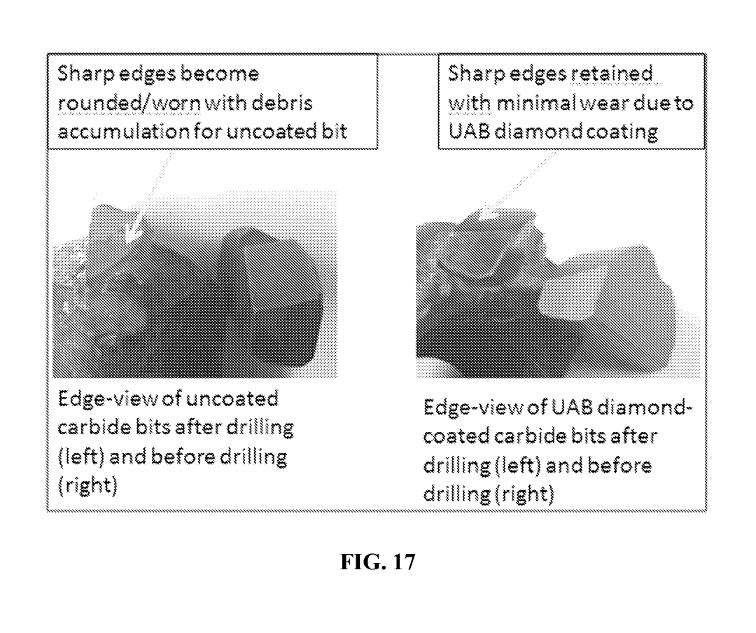

The tungsten carbide bits were used to drill holes in mines located in Birmingham, Ala. (considered “extremely hard rock conditions”) under normal production conditions (approximately 25 seconds per foot, no cooling). The holes are drilled for placement of roof bolts, used to maintain stability of the mine. It was found that the coated bits kept their speed and very little wear was observed compared to uncoated bits (FIGS. 16 and 17). The coated bits were compromised by fracture through the bulk carbide near the top center of bit.

Table 1 illustrates the average improvement in terms of linear feet drilled per trial for bits coated using the inventive methods described herein, as compared to traditional tungsten carbide. Each trial was comprised of multiple diamond coated samples.

TABLE 1
In-field rock mine drilling performance

| | | | Performance |
|---|---|---|---|
| Sample Type | Feet Drilled | Control | Increase |
| WC Roof | 13.5 | 6 | 225% |
| WC Roof | 66 | 21 | 314% |
| WC Roof | 93 | 18 | 517% |
| WC Roof | 56 | 36 | 156% |
| WC Roof | 250 | 50 | 500% |
| WC Roof | 100 | 40 | 250% |
| WC Roof | 108 | 36 | 300% |
| WC Roof | 108 | 36 | 300% |
| WC Roof | 90 | 25 | 360% |
| WC Roof | 132 | 42 | 314% |
| WC Directional | 4000 | 50 | 8,000% |
| WC Directional | 240 | 50 | 480% |
| WC Directional | 6000 | 50 | 12,000% |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A composite comprising:
a) a substrate;
b) a diamond film having a surface roughness of from about 5 nm to about 100 nm; and
c) an at least partially continuous metal boride layer disposed between a surface of the substrate and the diamond film.

2. The composite of claim 1, having a hardness of at least about 50 GPa.

3. (canceled)

4. The composite of claim 1, wherein the substrate comprises cobalt or an alloy thereof.

5. (canceled)

6. (canceled)

7. The composite of claim 1, wherein the substrate further comprises one or more of chromium, molybdenum, tungsten, titanium, aluminum, vanadium, nickel, iron, manganese, tungsten carbide, carbon, or a combination thereof.

8. The composite of claim 1, wherein the substrate comprises a metal carbide alloy.

9. The composite of claim 8, wherein the metal carbide alloy comprises one or more of tungsten carbide, titanium carbide, or a combination thereof.

10. The composite of claim 1, wherein the diamond film is positioned over at least a portion of the at least partially continuous metal boride layer.

11. (canceled)

12. The composite of claim 1, wherein the diamond film is substantially free of a graphitic carbon.

13. The composite of claim 1, wherein the diamond film comprises a nanostructured diamond film.

14. (canceled)

15. The composite of claim 1, wherein the diamond film is substantially free of an elemental metal.

16. (canceled)

17. (canceled)

18. The composite of claim 1, wherein the at least partially continuous metal boride layer comprises cobalt boride.

19. (canceled)

20. (canceled)

21. The composite of claim 1, wherein the at least partially continuous metal boride layer is conformal to the surface of the substrate.

22. The composite of claim 1, wherein the at least partially continuous metal boride layer is substantially free of elemental boron.

23. The composite of claim 18, wherein any cobalt present in the at least partially continuous metal boride layer is chemically bound to boron.

24. The composite of claim 18, wherein the at least partially continuous metal boride layer is substantially free of unbound cobalt.

25. The composite of claim 1, wherein the at least partially continuous metal boride layer has an average surface roughness from about 10 nm to about 75 nm.

26. The composite of claim 1, wherein the at least partially continuous metal boride layer exhibits an average hardness of from about 5 GPa to about 50 GPa.

27. A biomedical device comprising an orthopedic implant, wherein the biomedical device comprises the composite of claim 1.

28. (canceled)

29. A cutting device comprising a drill bit, wherein the cutting device comprise the composite of claim 1.

30. (canceled)

31. A film comprising an at least partially continuous metal boride layer that is conformal to a substrate and having an average surface roughness of from about 10 nm to about 75 nm.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)
